(19) United States
(12) Patent Application Publication
(10) Pub. No.: US 2022/0117205 A1
(43) Pub. Date: Apr. 21, 2022

(54) PORCINE SEXED SEMEN AND METHODS OF USE

(71) Applicant: ABS Global, Inc., DeForest, WI (US)

(72) Inventors: Matthew CULBERTSON, Wilmington, NC (US); Daniel HAMILTON, Wilmington, NC (US); Matthew EBERSOLE, Sun Prairie, WI (US); William HERRING, Gallatin, TN (US)

(21) Appl. No.: 17/501,716

(22) Filed: Oct. 14, 2021

Related U.S. Application Data
(60) Provisional application No. 63/092,299, filed on Oct. 15, 2020.

(51) Int. Cl. A01K 67/02

(52) U.S. Cl. CPC A01K 67/02; A01K 2227/108

(57) ABSTRACT

The present disclosure relates generally to methods for using porcine sex-sorted sperm cells for the efficient dissemination of desirable traits in multi-level swine production systems. The methods include using sex-sorted sperm cells for skewing offspring gender at the commercial farm level, producing porcine herds having improved growth performance traits, producing pathogen-resistant porcine herds, and disseminating desirable traits from a genetic nucleus to commercial farms using low dose artificial insemination techniques. The methods also provide a means for reducing costs at the production level by increasing the ratio of female offspring and improving animal welfare at all levels of production by reducing or eliminating male castration. In addition, the methods of the present technology may be employed to develop production flows for specialized pork products.

### CROSS-REFERENCE TO RELATED APPLICATIONS

This application claims the benefit of priority to U.S. Provisional Patent Application No. 63/092,299, filed Oct. 15, 2020, the entire contents of which are incorporated herein by reference.

### TECHNICAL FIELD

The present technology relates to methods of using porcine sex-sorted sperm cells for the efficient dissemination of desirable traits in multi-level swine production systems. More particularly, the present technology relates to methods of using sex-sorted sperm cells for skewing offspring gender at the commercial farm level and for the dissemination of pathogen-resistant markers and improved growth performance traits from a genetic nucleus to commercial farms in a few generations using artificial insemination techniques. The methods of the present technology also provide a means for reducing costs at the production level by increasing the ratio of female offspring and improving animal welfare at all levels of production by reducing or eliminating male castration. In addition, the methods of the present technology may be employed to develop production flows for specialized pork products.

### BACKGROUND

The following description of the background of the present technology is provided simply as an aid in understanding the present technology and is not admitted to describe or constitute prior art to the present technology.

The economic value of an animal as a parent for the next generation drives genetic improvement within the global livestock industry. In particular, the swine industry strives to select elite nucleus animals that would produce economically desirable offspring in a range of production systems (e.g., sow-housing systems, genetic nucleus, production nucleus, commercial farm) and environments (geographic and pathogen-free). The elite characteristic of a particular animal (i.e., its profit contribution) is determined by testing its offspring for growth, robustness, efficiency, and carcass value. Selection of desired traits and their dissemination in swine production have benefited from advances in molecular genetics, genomics, and computer sciences. As such, genetic improvement using molecular tools has become an important factor in the economic viability of pig production.

However, efficient swine production is limited by swine biology, driven by factors such as generation interval, genetic lag, and diseases. The generation interval is the average age of a parent when its offspring are born, and the genetic lag is the amount of time it takes to transmit a desired elite genetic trait from a nucleus population to commercial farms. In general, the generation interval for females is larger than that for males. Currently, artificial insemination, structured gilt development, and the replacement rate of gilt and sow allow the industry to maintain the average age of the parents when the offspring are born at a near optimum level. However, the amount of time it takes to get a particular elite genetic trait from the genetic nucleus to the commercial level remains a major impediment for the industry.

In the swine industry, traits are not created equal and they do not have the same economic importance in the global market. In addition, traits are measured in different units, and they have different heritability coefficients. The dissemination of a genetic trait from one generation to the next depends on the genetic superiority of the selected parents. The genetic superiority of a particular individual depends on the heritability of the trait, and the relationship between the performance of the selected parents and the swine herd. Currently, the estimated breeding value (EBV) of a particular parent based on the performance of its offspring is used to minimize the genetic lag between the genetic nucleus and target herd. EBV is measured using commercially available methods such as Best Linear Unbiased Prediction (BLUP). BLUP estimates EBV by removing phenotypic superiorities caused by environmental factors while simultaneously using additive genetic relationships between animals. However, BLUP cannot correct for environmental factors when animals in different environments are not related. Accordingly, there is a need for improved methods for minimizing the genetic lag between generations to increase the dissemination of desirable traits in swine production.

Genetic lag is closely linked to the underlying biology of swine reproduction and parturition. In particular, the sex ratio of a pregnancy has been difficult to control, and the generation of a large proportion of an undesirable sex (e.g., males) can be economically disadvantageous. In the swine industry, a large proportion of male offspring may not be desirable at the commercial level due to issues associated with boar taint. Current methods of addressing the boar taint issues at the commercial production level include: surgical castration within three days of life; immuno-castration using FDA-approved veterinary vaccines such as Improvest®; chemical castration, which involves local destruction of testicular tissue with chemicals such as lactic acid or zinc salts; slaughtering boars before they reach puberty; and genetic selection for boars with low boar taint. These methods are not currently economically efficient because some of them pose animal welfare issues, while others require high operational costs of breeding and commercial farms. Accordingly, there is a need to pre-select animal offspring gender to skew commercial lines toward the production of a higher percentage of female offspring.

Sex-sorting sperm when coupled with genetic trait selection and dissemination has the potential of reducing the genetic lag in swine production.

### SUMMARY

In one aspect, the present disclosure provides a method for producing a pathogen-resistant porcine herd or population, comprising: inseminating one or more target sows with a sex-sorted sperm cell sample from a boar, wherein the boar comprises one or more pathogen-resistant markers, thereby producing offspring comprising one or more pathogen-resistant markers.

In some embodiments, the method further comprises inseminating one or more females from the offspring with a sex-sorted sperm cell sample from a boar having one or more pathogen-resistant markers, wherein the one or more pathogen-resistant markers from the boar and the one or more female offspring are the same or different.

In some embodiments, the one or more female offspring is a member of a daughter nucleus line or a multiplier line, and the boar is a member of a genetic nucleus line.

In some embodiments, inseminating the one or more target sows comprises administering the sex-sorted sperm cell sample to a reproductive tract of the one or more target sows using intra-cervical insemination (ICAI), intrauterine insemination (IUI), deep intrauterine insemination (DIUI), or intratubal insemination (ITI).

In some embodiments, the boar is a member of a genetic nucleus line, a daughter nucleus line or a multiplier line.

In some embodiments, the one or more target sows is a member of a genetic nucleus line, a daughter nucleus line, or a multiplier line.

In some embodiments, the pathogen is selected from the group consisting of porcine reproductive and respiratory syndrome (PRRS) virus, Escherichia coli F18, Escherichia coli F4ab, Escherichia coli F4ac, circovirus, swine influenza virus, coronavirus, Mycoplasma hyopneumoniae, and Actinobacillus pleuropneumoniae.

In some embodiments, the pathogen-resistant herd or population is resistant to a pathogen selected from the group consisting of porcine reproductive and respiratory syndrome (PRRS) virus, Escherichia coli F18, Escherichia coli F4ab, Escherichia coli F4ac, circovirus, swine influenza virus, coronavirus, Mycoplasma hyopneumoniae, and Actinobacillus pleuropneumoniae. In some embodiments, the pathogen is a porcine reproductive and respiratory syndrome (PRRS) virus.

In some embodiments, the one or more pathogen-resistant markers comprise one or more mutations in a gene selected from the group consisting of alpha (1,2) fucosyltransferase 1 (FUT1), Mucin 4, myxovirus resistance protein 1 (Mx1), HLA-B associated transcript 2 (BAT2), Euchromatic histone-lysine N-methyltransferase 2 (EHMT2), Aminopeptidase N (ANPEP), Acidic nuclear phosphoprotein 32 family member A (ANP32A), ANP32B, transmembrane serine protease 2 (TMPRSS2), transmembrane serine protease 4 (TMPRSS4), CD163, and sialic acid binding Ig-like lectin 1 (SIGLEC1). In some embodiments, the one or more mutations comprise insertions, deletions, substitutions, or a combination thereof.

In some embodiments, the sex-sorted sperm cell sample from the boar comprises a combination of sex-sorted sperm cells from one or more boars having one or more pathogen-resistant markers.

In some embodiments, the sex-sorted sperm cell sample has a concentration of between about 2×10⁴ to about 4×10⁶ sperm cells per milliliter.

In one aspect, the present disclosure provides a method of enhancing the dissemination of improved growth performance traits in a porcine herd or population, comprising: (a) obtaining a sperm cell sample from a boar, wherein the boar comprises one or more improved growth performance traits selected from the group consisting of: enhanced growth efficiency, enhanced meat quality, enhanced reproductive quality, and improved health; (b) enriching the sperm cell sample obtained from the boar; (c) inseminating one or more target sows with the enriched sperm cell sample; and (d) producing offspring having the one or more improved growth performance traits.

In some embodiments, the method further comprises: (e) selecting one or more female offspring having the one or more improved growth performance traits that would enhance the dissemination of the one or more improved growth performance traits to the next generation; and (f) inseminating the selected one or more female offspring with an enriched sperm cell sample that was obtained from a sperm cell sample of a boar having one or more improved growth performance traits, wherein the one or more improved growth performance traits from the boar and the one or more female offspring are the same or different.

In some embodiments, the enhanced growth efficiency trait is selected from the group consisting of increased average daily gain, increased average daily feed intake, increased feed efficiency, reduced back fat thickness, increased muscle mass, increased loin muscle area, and increased carcass lean percentage.

In some embodiments, the enhanced growth efficiency trait comprises a mutation in a gene encoding a protein selected from the group consisting of ryanodine receptor, protein kinase AMP-activated gamma 3 (AMPKy-3, PRKAG3)), paired-like homeodomain transcription factor 2 (Pitx2), Insulin-like growth Factor 2 (IGF2), high mobility group AT-hook 2 (HMG2A), cholecystokinin A receptor (CCKAR), fatty acid synthase (FASN), calpastatin (CAST 249, 638), and melanocortin-4 receptor (MC4R) gene. In some embodiments, the improved growth performance trait comprises an enhanced reproductive quality and wherein the enhanced reproductive quality comprises a mutation in a gene encoding a protein selected from the group consisting of estrogen receptor (ER) and erythropoietin receptor (EPOR).

In some embodiments, inseminating the one or more target sows comprises administering the sex-sorted sperm cell sample to a reproductive tract of the one or more target sows using intra-cervical insemination (ICAI), intrauterine insemination (IUI), deep intrauterine insemination (DIUI), or intratubal insemination (ITI).

In some embodiments, the boar is a member of a genetic nucleus line, a daughter nucleus line, or a multiplier line.

In some embodiments, the one or more target sows are members of a genetic nucleus line, a daughter nucleus line, or a multiplier line.

In some embodiments, the one or more female offspring are members of a daughter nucleus line, or a multiplier line, and the boar is a member of a genetic nucleus line.

In some embodiments, the one or more mutations comprise insertions, deletions, substitutions, or a combination thereof.

In some embodiments, the enriching of the sperm cell sample obtained from the boar comprises sexing the sperm cell sample for X- or Y-chromosome bearing sperm cells to obtain a sex-sorted sperm cell sample.

In some embodiments, the sex-sorted sperm cell sample from the boar comprises a combination of sex-sorted sperm cells from one or more boars having one or more improved growth performance traits.

In some embodiments, the sex-sorted sperm cell sample has a concentration of between about 2×10⁴ to about 4×10⁶ sperm cells per milliliter.

In one aspect, the present disclosure provides a method of increasing the number of female offspring in a porcine herd or population, comprising: inseminating one or more target sows with a sex-sorted sperm cell sample from a boar to produce offspring, wherein the boar is a member of a genetic nucleus line, and the one or more target sows are members of a daughter nucleus line or a multiplier line, and wherein about 65% to about 99% of the offspring are female.

In some embodiments, the offspring are terminal parent lines.

In some embodiments, the inseminated one or more target sows produce about 0 to 35% male offspring.

In some embodiments, the sex-sorted sperm cell sample comprises at least about 65%, at least about 70%, at least about 75%, at least about 80%, at least about 85%, at least about 90%, at least about 95%, at least about 96%, or at least about 99% X-chromosome bearing sperm cells.

In some embodiments, inseminating the one or more target sows comprises administering the sex-sorted sperm cell sample to a reproductive tract of the one or more target sows using intra-cervical insemination (ICAI), intrauterine insemination (IUI), deep intrauterine insemination (DIUI), or intratubal insemination (ITI).

In some embodiments, the sex-sorted sperm cell sample has a concentration of between about 2×10⁴ to about 4×10⁶ sperm cells per milliliter.

In some embodiments, inseminating the one or more target sows comprises administering at least 0.5×10⁶ sex-sorted sperm cells to the reproductive tract of the one or more target sows using intratubal insemination.

In some embodiments, inseminating the one or more target sows comprises administering at least 10×10⁶ sex-sorted sperm cells to the reproductive tract of the one or more target sows using deep intrauterine insemination.

In some embodiments, the sex-sorted sperm cell sample is fresh or cryopreserved.

In one aspect, the present disclosure provides a method of producing a pathogen-resistant female porcine herd or population, comprising: inseminating one or more female target sows with a sex-sorted sperm cell sample from an elite boar, wherein at least 60% of the sperm cells in the sex-sorted sperm cell sample carry X chromosomes, and wherein the boar comprises one or more pathogen-resistant markers, to produce offspring, wherein about 65% to about 99% of the offspring are female, and the female offspring comprise the one or more pathogen-resistant markers.

In some embodiments, that method further comprises: inseminating one or more females from the offspring with a sex sorted sperm cell sample from the boar having one or more pathogen-resistant markers, wherein the one or more pathogen-resistant markers from the boar and the one or more female progeny are the same or different, and wherein at least 60% of the sperm cells in the sex-sorted sperm cell sample carry X chromosomes.

In some embodiments, the one or more female progeny are members of a daughter nucleus line, or a multiplier line, and the boar is a member of a genetic nucleus line, or a daughter nucleus line.

In some embodiments, inseminating the one or more target sows or one or more females from the offspring comprises administering the sex sorted sperm cell sample to a reproductive tract of the one or more target sows using intra-cervical insemination (ICAI), intrauterine insemination (IUI), deep intrauterine insemination (DIUI), or intratubal insemination (ITI).

In some embodiments, the boar is a member of a genetic nucleus line, a daughter nucleus line, or a multiplier line.

In some embodiments, the one or more target sows is a member of a genetic nucleus line, a daughter nucleus line, or a multiplier line.

In some embodiments, the pathogen is selected from the group consisting of porcine reproductive and respiratory syndrome (PRRS) virus, Escherichia coli F18, Escherichia coli F4ab, Escherichia coli F4ac, circovirus, swine influenza virus, Mycoplasma hyopneumoniae, and Actinobacillus pleuropneumoniae.

In some embodiments, the pathogen-resistant herd or population is resistant to a pathogen selected from the group consisting of porcine reproductive and respiratory syndrome (PRRS) virus, Escherichia coli F18, Escherichia coli F4ab, Escherichia coli F4ac, circovirus, swine influenza virus, Mycoplasma hyopneumoniae, and Actinobacillus pleuropneumoniae. In some embodiments, the pathogen is a porcine reproductive and respiratory syndrome (PRRS) virus.

In some embodiments, the one or more pathogen-resistant markers comprise one or more mutations in a gene selected from the group consisting of alpha (1,2) fucosyltransferase 1 (FUT1), Mucin 4, myxovirus resistance protein 1 (Mx1), HLA-B associated transcript 2 (BAT2), euchromatic histone-lysine N-methyltransferase 2 (EHMT2), Aminopeptidase N (ANPEP), Acidic nuclear phosphoprotein 32 family member A (ANP32A), ANP32B, transmembrane serine protease 2 (TMPRSS2), transmembrane serine protease 4 (TMPRSS4), CD163, and sialic acid binding Ig-like lectin 1 (SIGLEC1)

In one aspect, the present disclosure provides a method of producing an enhanced female porcine herd or population with one or more improved growth performance traits, comprising: inseminating one or more target sows with a sex-sorted sperm cell sample from an elite boar, wherein at least 60% of the sperm cells in the sex-sorted sperm cell sample carry X chromosomes, and wherein the boar has one or more improved growth performance traits, to produce offspring, wherein about 65% to about 99% of the offspring are female, and the female offspring carry the one or more improved growth performance traits.

In some embodiments, the one or more improved growth performance traits is selected from: enhanced growth efficiency; enhanced meat quality; enhanced reproductive quality; and improved health.

In some embodiments, the one or more improved growth performance traits is an enhanced growth efficiency trait selected from the group consisting of: increased average daily gain; increased average daily feed intake; increased feed efficiency; reduced back fat thickness; increased muscle mass; increased loin muscle area; and increased carcass lean percentage.

In some embodiments, the offspring are terminal parent lines.

In some embodiments, the inseminated one or more target sows produce about 0 to 35% male offspring.

In some embodiments, the sex-sorted sperm cell sample comprises at least about 65%, at least about 70%, at least about 75%, at least about 80%, at least about 85%, at least about 90%, at least about 95%, at least about 96%, or at least about 99% X-chromosome bearing sperm cells.

In some embodiments, the enhanced growth efficiency trait comprises a mutation in a gene encoding a protein selected from the group consisting of ryanodine receptor, protein kinase AMP-activated gamma 3 (AMPKy-3, PRKAG3)), paired-like homeodomain transcription factor 2 (Pitx2), insulin-like growth factor 2 (IGF2), high mobility group AT-hook 1 (HMG1A), cholecystokinin A receptor (CCKAR), fatty acid synthase (FASN), calpastatin (CAST 249, 638), and melanocortin-4 receptor (MC4R).

In one aspect, the present disclosure provides a method for producing a high health porcine herd or population comprising inseminating one or more sows with a sex-sorted sperm cell sample from a boar that is selected for having at least one health trait to produce a progeny having high health.

In some embodiments, the health trait is selected from one or more of: the absence of an undesirable physical abnormality; improved feet and leg soundness; resistance to specific diseases or disease organisms; or general resistance to pathogens.

In some embodiments, the undesirable physical abnormality is selected from one or more of: a propensity for inguinal hernia; cryptorchidism; atresia ani; and splay leg.

In some embodiments, the pathogen is selected from one or more of porcine reproductive and respiratory syndrome (PRRS) virus, Escherichia coli F18, Escherichia coli F4ab, Escherichia coli F4ac, circovirus, swine influenza virus, Mycoplasma hyopneumoniae, and Actinobacillus pleuropneumoniae. In some embodiments, the pathogen is PRRS virus.

In some embodiments, the resistance to specific disease organisms is associated with one or more mutations in a gene selected from the group consisting of alpha (1,2) fucosyltransferase 1 (FUT1), Mucin 4, myxovirus resistance protein 1 (Mx1), HLA-B associated transcript 2 (BAT2), Euchromatic histone-lysine N-methyltransferase 2 (EHMT2), Aminopeptidase N (ANPEP), Acidic nuclear phosphoprotein 32 family member A (ANP32A), ANP32B, transmembrane serine protease 2 (TMPRSS2), transmembrane serine protease 4 (TMPRSS4), CD163, and sialic acid binding Ig-like lectin 1 (SIGLEC1).

In some embodiments, the one or more mutations comprise insertions, deletions, substitutions, or a combination thereof.

In some embodiments, the specific disease is Rendement Napole or porcine stress syndrome.

In some embodiments, the sex-sorted sperm cell sample from the boar comprises a combination of sex-sorted sperm cells from one or more boars having at least one health trait.

In some embodiments, the sex-sorted sperm cell sample has a concentration of between about 2×10⁴ to about 4×10⁶ sperm cells per milliliter.

In some embodiments, the sex-sorted sperm cell sample used in any of the methods described above comprises any of the amounts or percentages of X- or Y-chromosome bearing sperm cells as set forth herein.

### BRIEF DESCRIPTION OF THE DRAWINGS

FIG. 1A is a schematic showing a general outline of a method for processing boar semen to obtain a porcine sexed semen sample.

FIG. 1B is a chart showing the X-skew % of an enriched semen sample processed by the methods described herein. The sample was processed on a flow cytometer at a rate of 17,500 cells/second. The ejaculate sample was obtained from Boar 9022.

### DETAILED DESCRIPTION

It is to be appreciated that certain aspects, modes, embodiments, variations and features of the present methods are described below in various levels of detail in order to provide a substantial understanding of the present technology. The definitions of certain terms as used in the specification are provided below. Unless defined otherwise, all technical and scientific terms used herein generally have the same meaning as commonly understood by one of ordinary skill in the art to which this present technology belongs.

The present technology is directed to methods of producing pathogen-resistant and improved growth performance traits in a herd or line of swine by inseminating sow lines using sex-sorted sperm cell samples, and efficiently disseminating these traits from a genetic nucleus to commercial farms. The use of sex-sorted sperm cell samples as disclosed herein would permit one of ordinary skill in the art to disseminate transgenic or modified swine lines carrying one or more desirable traits to commercial production in a cost effective and/or profitable manner. In some embodiments, the present technology provides methods for creating desirable swine products using very few or a single boar to inseminate an entire generation of female targets without sacrificing long-term breeding and selection programs that occur at the genetic nucleus level.

### I. Definitions

As used herein, the singular forms “a,” “an,” and “the” designate both the singular and the plural, unless expressly stated to designate the singular only

As used herein, the term “about” and the use of ranges in general, whether or not qualified by the term about, means that the number comprehended is not limited to the exact number set forth herein, and is intended to refer to ranges substantially within the quoted range while not departing from the scope of the present technology. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein “commercial swine” refers to swine slaughtered for their meat for commercial sale or sows producing swine for their meat for commercial sale. “Commercial farm,” as used herein, refers to a facility for housing commercial swine.

As used herein, the term “daughter nucleus” refers to one or more populations of male and female swine that is of the same genetic line as the genetic nucleus line, and is used to transfer the genetic improvement issued from the genetic nucleus to the multiplier line through breeding.

As used herein, the terms “efficient growth traits” and/or “performance traits” refer to a group of traits that are related to growth rate and/or body composition of the animal. Examples of such traits include, but are not limited to, average daily gain, average daily feed intake, feed efficiency, back fat thickness, loin muscle area, and lean percentage. In some embodiments, pigs produced by methods of the present technology have one or more performance trait genes.

As used herein, an “elite boar” is a boar produced in the genetic nucleus herd that has extremely high breeding value, such as superior genetic potential relative to the mean of the genetic nucleus herd population.

As used herein, the term “estimated breeding value” (EBV) refers to a specific numeric value for an animal that predicts its “breeding value.” EBV is often calculated using commercially available analysis programs (the output from BLUP and marker assisted BLUP programs are examples of EBV's). Calculation of EBVs is known in the art (see, e.g., Misztal et al., Computing procedures for genetic evaluation including phenotypic, full pedigree, and genomic information, J. Dairy Sci. 92:4648-4655 (2009), which is herein incorporated by reference).

As used herein, the terms “genetic nucleus” and “genetic nucleus herd” refer to one or more populations of male and female swine that are used for increasing the genetic improvement of the one or more lines, and serve as the source of genetic improvement over time. Top ranking young males and females are identified from this population each generation and used in the genetic nucleus herd to replace older, lower ranking animals thereby creating genetic improvement that accumulates from one generation to the next.

As used herein the term “high health” is meant to refer to a herd or population of animals that is characterized by the absence of certain diseases. It is a relative term. For example, in one embodiment of the present technology, “high health” refers to the target herd from which terminal boars are sold. Such boars are free of certain pathogens or diseases, such as, e.g., porcine respiratory and reproductive syndrome (PRRS) or Mycoplasma pneumonia (Mycoplasma hyopneumoniae). The genetic nucleus herds may not be free of these pathogens or diseases.

As used herein, the term “improved growth performance trait gene” refers to genes, alleles, variants, or genetic markers that correlate with enhanced growth efficiency, enhanced meat quality, enhanced reproductive quality, and/or improved health.

As used herein, the terms “line” and “breed” refer to a group of animals having a common origin and similar identifying characteristics. In some embodiments, the present technology is applicable to “pure lines,” “pure breeds,” and “crossbreeds” of swine, as those terms are used in the art.

As used herein, the term “marker” refers to a sequence of DNA that has a specific location on a chromosome that can be measured in a laboratory. To be useful, a marker needs to have two or more alleles. Common types of markers include, but are not limited to restriction fragment length polymorphism (RFLP), simple sequence repeat (SSR; “microsatellite” markers), and single nucleotide polymorphism (SNP). Markers can be either direct, that is, located within the gene or locus of interest, or indirect, that is, closely linked with the gene or locus of interest (presumably due to a location which is proximate to, but not inside the gene or locus of interest).

As used herein, the term “marker assisted allocation” (MAA) refers to the use of phenotypic and genotypic information to identify animals with superior estimated breeding values (EBVs) and the further allocation of those animals to a specific use designed to improve the genetic merit of terminal boars for sale or to improve the genetic nucleus or target herds. In some embodiment, the MAA is used for the selection of multiple traits and for balancing each trait based on its economic value and heritability. In some embodiments, the present disclosure provides a means of using markers to identify swine lines suitable for use according to the methods of the present technology. In some embodiments, selected swine lines are allocated for use to most effectively and efficiently bring about the desired genetic improvements in offspring animals using MAA.

As used herein, the term “marker-assisted selection” (MAS) refers to the use of phenotypic and genotypic information to identify animals with superior estimated breeding values (EBVs) for selection and use as breeding animals for the genetic nucleus herd.

As used herein, the term “multiplier,” or “multiplication unit,” or “multiplier herd” refers to one or more populations of male and female swine that are used to propagate and increase the number of male and female swine with the genetic improvement issued from the genetic nucleus. These lines may be pure line or crossbred products, and are used as parents, grandparents, or great grandparents of commercial swine.

As used herein, the term “selection index” refers to a numerical score generated for an individual swine breed based on the swine's expression of certain traits selected by a breeder. Generally, a breeder would calculate estimated breeding values for each economic trait for each animal based on pedigree and phenotypic information such as phenotypic records on ancestors, offspring; and the animal itself.

As used herein, the term “sire line” refers to a line that contributes to the production of parent boars used on commercial farms. Also, as used herein. “dam line” refers to a line that contributes to the production of parent gilts/sows used on commercial farms. At every level in the swine production pyramid, for each mating type, from genetic nucleus to commercial production, one has males (boars and/or their semen) and females (gilts/sows and/or their eggs) to choose from for the production of progeny.

As used herein, the term “sow” generally refers to any female swine, including sows and gilts. A sow may be a member of a genetic nucleus, a multiplier herd, or a commercial farm.

As used herein, the term “swine production herd” or “production herd” or “commercial swine” refers to a collection of animals whose primary purpose is to produce pigs that will be slaughtered for their meat for commercial sale.

As used herein, a “target herd” refers to a non-genetic nucleus herd of female swine to which elite boars from the genetic nucleus herd are mated to for the purpose of transferring desirable genes/traits from the genetic nucleus to the target herd. In some embodiments, the target herd is made up of purebred females from the same genetic line as that of the genetic nucleus herd or may be made up of any swine females for which it would be desirable to include desirable traits from the genetic herd. In some embodiments, the target herd is a daughter nucleus herd or a multiplier herd. In some embodiments, the target herd is a crossbreed herd.

### II. Uses of Sex-Sorted Porcine Sperm Cells

The present disclosure provides for the use of sex-sorted porcine sperm cells to increase the dissemination of one or more desirable traits in a porcine line or breed, such that a high percentage of offspring will express the one or more desirable traits, and the penetrance of the one or more traits will be higher in the offspring than in the parent or grandparent generations.

A. Sex-Sorted Porcine Sperm Cells

In some embodiments, a sperm cell sample obtained from one or more boars is enriched. In some embodiments, the enrichment comprises sexing the sperm cell sample for X- or Y-chromosome bearing sperm cells to obtain a “sex-sorted” (also referred to as a “sex-selected” or “skewed” or “enriched”) sperm cell sample. In some embodiments, the sex-sorted porcine sperm cells have undergone one or more selections. In some embodiments, sperm cells may be selected for use based on sex-type. Sex-sorted sperm cell samples from boars with desired genetic traits provide swine breeders with the opportunity to advance herd genetics and therefore farm profitability, while ensuring that the offspring predominately of the desired sex are born in the next generation. In addition, the capability to pre-select animal offspring gender will allow more efficient operations for livestock producers.

Sex-sorted porcine sperm samples can be produced according to methods known in the art. Sex-selection methods include magnetic techniques (see, e.g., U.S. Pat. No. 4,276,139), columnar techniques (see, e.g., U.S. Pat. No. 5,514,537) and gravimetric techniques (see, e.g., U.S. Pat. Nos. 4,092,229, 4,067,965, and 4,155,831). Sex selection based on differences in electrical properties is disclosed in U.S. Pat. No. 4,083,957, and techniques that select based on differences in electrical and gravimetric properties are discussed in U.S. Pat. Nos. 4,225,405, 4,698,142, and 4,749,458. U.S. Pat. Nos. 4,009,260, and 4,339,434 describe selection based on differences in motility. Biochemical techniques relying on antibodies are disclosed in U.S. Pat. Nos. 4,511,661, 4,999,283, 4,191,749, and 4,448,767. U.S. Pat. Nos. 5,021,244, 5,346,990, 5,439,362, and 5,660,997 describe selection based on differences in membrane proteins. U.S. Patent Publication Nos. 2013/0007903 and 2002/0119558, U.S. Pat. Nos. 4,362,246, 5,135,759, 5,150,313, 5,602,039, 5,602,349, and 5,643,796, and International Patent Application Publication No. WO 1996/012171 provide sperm cell sorting techniques using flow cytometry. U.S. Patent Publication No. 2003/0157475 provides methods for cryopreserving selected sperm and maintaining fertility. Each of the foregoing references is hereby incorporated by reference in its entirety.

Many methods have been attempted to achieve the separation of X- and Y-chromosome bearing sperm. These methods have ranged from magnetic techniques such as appears disclosed in U.S. Pat. No. 4,276,139 to columnar techniques as appears disclosed in U.S. Pat. No. 5,514,537 to gravimetric techniques as discussed in U.S. Pat. No. 3,894,529, reissue U.S. Pat. No. 32,350, U.S. Pat. Nos. 4,092,229, 4,067,965, and 4,155,831. Electrical properties have also been attempted as shown in U.S. Pat. No. 4,083,957 as well as a combination of electrical and gravimetric properties as discussed in U.S. Pat. Nos. 4,225,405, 4,698,142, and 4,749,458. Motility efforts have also been attempted as shown in U.S. Pat. Nos. 4,009,260 and 4,339,434. Chemical techniques such as those shown in U.S. Pat. Nos. 4,511,661 and 4,999,283 (involving monoclonal antibodies) and U.S. Pat. Nos. 5,021,244, 5,346,990, 5,439,362, and 5,660,997 (involving membrane proteins), and U.S. Pat. Nos. 3,687,803, 4,191,749, 4,448,767, and 4,680,258 (involving antibodies) as well as the addition of serum components as shown in U.S. Pat. No. 4,085,205. While each of these techniques has been presented as if to be highly efficient, in fact at present none of those techniques yield the desired level of sex preselection. Each of the foregoing references is hereby incorporated by reference in its entirety.

In some embodiments, any one or more the following approaches is used to obtain sexed porcine semen: Sex-linked motility inhibition using TLR7/8 agonists (e.g., TLR7/8 genes are on the X-chromosome and expression may be enriched in X-chromosome containing sperm cell; TLR7/8 agonists may cause sex-specific changes in motility that may allow for the separation of X and Y containing cells in a swim up); WholeMom Antibody Mediated Agglutination of Y-chromosome bearing cells (e.g., use of an antibody that specifically agglutinates Y-chromosome containing sperm cells, allowing for the enrichment of X-chromosome containing cells); Hypersensitization/Immune stimulation against male embryos to favor female births (female side implementation); SRY transcript/protein differential expression in X and Y containing sperm cells (e.g., SRY transcript enrichment in Y-chromosome bearing sperm cells as a target for antibody or genetic targeting of Y-chromosome bearing sperm cells); H-Y Antigen enriched expression on Y-chromosome cells; Candidate protein/pathway approach (e.g., identify compounds that selectively target proteins or pathways published as differentially expressed in X- and Y-sperm and determine their potential in sexing application); Natural, competitive selection (e.g., capitalize on mechanism that results in competitive selection of X-bearing sperm cells in natural breeding settings); Panning of Recombinant Antibody Libraries to Identify antibodies that can discriminate between X and Y cells (e.g., use whole cell (x or y specific populations) or candidate antigens (discovered from target identification screening) to pan and counter-select for antibodies that bind cells in a sex-dependent manner; deep mining of phage display libraries may uncover rare antibodies binding previously unknown epitopes); Screening for sex-specific binding aptamers (e.g., use whole cells, lysates, or candidate proteins to probe aptamer libraries for specific affinity binding reagents that will allow for the use of magnetic beads or column chromatography to bulk sex semen); Compound and/or Toxin library screen for X- and Y-selectivity (e.g., screen compound libraries (particularly targeting surface proteins) and/or toxin libraries for sex-selection by quantifying binding to X-enriched vs. Y-enriched semen); 10× Chromium single cell expression analysis to identify differentially expressed targets in X and Y cells (e.g., use single cell expression profiling to identify differentially expressed gene targets in X and Y sperm cells that may be attractive targets for either pharmacological or affinity-based sex-separation approaches); miRNA profiling; CITE-Seq or REAP-Seq, JESS to identify differentially expressed RNA/Protein targets on X and Y-Sperm, or male and female embryos (e.g., CITE-seq (cellular indexing of transcriptomes and epitopes by sequencing) and REAP-seq (RNA expression and protein sequencing assay), or a capillary Western blot assay like JESS can allow for the identification of protein markers preferentially or specifically expressed on X- or Y-chromosome bearing sperm or male and female embryos); Global and/or surface Quantitative Proteomic Analysis of Y- and X-chromosome bearing sperm cells from multiple sires (e.g., discovery of differentially expressed proteins in X and Y porcine sperm cells to identify targets for both biochemical and genetic-based bulk processing strategies); Hoechst-derivatives for increasing efficiency of sorting X- and Y-chromosome bearing sperm (e.g., using visible light to detect bovine X and Y-bearing sperm populations); Production of an on/off detection system for differentiating X and Y sperm; Guided design of Pyrrole-Imidazole Polyamide small molecule DNA binding molecules for specific X- or Y-chromosome binding; SmartFlare™ approach to target gender-specific RNA transcripts (e.g., use RNA transcripts that are sex-specific targets for SmartFlare™ probes designed to fluoresce in live cells); Split MiniSOG light inducible cell death (e.g., MiniSOG is a small (106 aa) protein that belongs to a broad family of optogenetic tools for inducing cell death inducible by light exposure); Deliver molecules to sperm cells (e.g., deliver otherwise cell impermeant molecules to sperm cells); Posttranslational Modifications Effects on Male Fertility and Sperm Viability (e.g., differential PTM (SUMOylation, ubiquitination, phosphorylation, acetylation, glycosylation) characterization in male and female sperms exploit that difference to design the sorting procedure); Marker integration on Y chromosome (e.g., integrate a marker, such as fluorescence, antibiotic, surface protein, on the Y chromosome); STAGE (sperm transfection assisted gene editing) (e.g., deliver a nuclease (+/−a transgene cassette) via lipofection or electroporation; expression of components would be designed to impact development of male embryos); Nanoparticle-based transformation of germ cells (e.g., use of nanoparticles to deliver exogenous DNA constructs to germ cells in vivo, directly targeting differentiating cells and sperm cells in sires; either target developing spermatids or mature sperm cells to alter the sex ratio of fertilization capable sperm cells); Targeted disruption of SRY testis-determining transcription factors (e.g., loss of function mutants for SRY proteins (pigs have duplicated SRY genes) produce animals that are phenotypically female); CRISPR-Cas9 induced self destruction of male embryos (e.g., constitute expression of Y-linked guide RNA (sgRNA) targeting essential genes in male and constitutive expression of Cas9 in female; male specific inheritance of guide RNA via sperm and female specific expression of Cas9 in oocytes will result knockout of essential gene in only male embryos and will lead to embryonic death (CRISPR-Cas9 bi-component)); Dual component rescue of development (e.g., a transgene system with one element (e.g., microRNA on Y chromosome) acting to inhibit a developmentally required gene, with a rescue cassette expressing the required gene (on X or autosome)); Y-spermatid specific lethal gene expression under the promoters/signal peptide of cytoplasmic bridge resistant gene/protein (e.g., in mice, some genes like smoke2a/2b, Spaml, Smpdl, TLR7/8 have been reported to escape cytoplasmic bridge; using promoter of these genes or signal peptide of these proteins can be used to express either X spermatozoa specific suicide gene or induce the imbalance expression of any protein that can be exploited in sperm sorting); Y-chromosome meddling using a Y-chromosome repeat sequence targeted guide RNA and Cas9 (e.g., use gRNA directed against Y-chromosome specific repeat sequences to target Cas9 and potentially a tethered chromatin remodeling factor to either fragment the Y chromosome or perturb chromatin structure sufficiently to induce physiological changes in Y-chromosome containing sperm cells); Bimolecular complementation development inhibition (e.g., develop a two component system of inhibiting development (e.g., yeast two-hybrid, ZFN or TAL left and right binding site with KRAB domain) one of which would be encoded on the Y chromosome of the male line and the other in the female line as homozygous either on X or autosomal; when both components are together in a male embryo they would act to inhibit a developmental gene); Separation of X and Y Cells using Zeta Potential and a charged flow device (e.g., detect differences in zeta potential between X- and Y-chromosome bearing cells); Density Differences (e.g., due to the DNA content difference in the X- and Y-chromosome bearing cells, there may exist a difference in density between the two sexes of sperm cells); Mass Differences (e.g., distinguishing between X- and Y-chromosome bearing sperm based on the difference in their DNA mass); Motility Difference (e.g., distinguishing between X- and Y-chromosome bearing sperm based on the difference in their motility); Temperature and Heat Stress (e.g., a differential response to heat or pH stress between the two different sexes of sperm may enable separation through a parameter like motility or expression of a surface protein); Sephadex Gel Separation (e.g., filtration of prepared ejaculates through a sephadex gel column could produce an enrichment in sex (detected in the retained cells in the column and the filtrate); mechanistic explanation for the occurrence is unclear but could be linked to motility or absorption/adsorption parameters); Heavy Metal Absorption (e.g., heavy metal exposure to males (sperm cells) may enable a selection mechanism via a toxicity to one sex of gametes or via differential absorption into or adsorption onto the cell); Life of sperm (e.g., timing of insemination vs. ovulation to impact the sex ratio); and Size based separation of X- and Y-chromosome bearing sperm cells.

In some embodiments, distinguishing male-producing from female-producing sperm cells is accomplished by exploiting the difference in total DNA content between the X- and Y-chromosomes. In particular, the difference in total DNA between X-bearing sperm and Y-bearing sperm cells is about 3.4%. Generally, the X chromosome contains more DNA than does the Y chromosome. Commercial sex-sorted sperm cell samples are currently produced by staining sperm cells with Hoechst 33342, a dye that penetrates live cells, binds stoichiometrically to DNA, and releases a fluorescent signal when excited. This fluorescent signal allows a flow cytometer to quantitatively discriminate X- and Y-chromosome-containing sperm cells based on the difference in total DNA content. Sperm cells may then be segregated into separate containers based on the presence or absence of a particular sex chromosome and/or the cells of the undesired sex may be laser-ablated to produce a sex sorted sperm cell sample. The utility of sexed semen depends upon the percentage of X- and Y-chromosome-bearing sperm present in the product, which in turn depends on the accuracy of both making sex-sorted sperm cells and verifying the final sex skew.

In some embodiments, the sex-skewed population of sperm cells comprises an X-chromosome in at least about 60%, at least about 65%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, or at least about 97% of the sperm cells in the population. In some embodiments, the sex-skewed population of sperm cells comprises a Y-chromosome in at least about 60%, at least about 65%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, or at least about 97% of the sperm cells in the population. In some embodiments, the sex-skewed population is confirmed to bear an X-chromosome in between 60 and 75%, between 75 and 80%, between 80 and 85%, between 85 and 90%, between 90 and 95%, and between 95 and 97% of the sperm cells in the population. In some embodiments, the sex-skewed population is confirmed to bear a Y-chromosome in between 60 and 75%, between 75 and 80%, between 80 and 85%, between 85 and 90%, between 90 and 95%, and between 95 and 97% of the sperm cells in the population.

B. Artificial Insemination

In some embodiments, the present technology relates to insemination of a target sow by employing sperm cell samples sorted by one of the methods described above. Once a sex-sorted sperm cell sample has been assessed, the sperm cell population may be used to inseminate a target sow. Insemination may be performed according to any number of methods known to those of skill in the art. These methods include conventional artificial insemination techniques such as intra-cervical insemination (ICAI), intrauterine insemination (IUI), deep intrauterine insemination (DIUI), or intratubal (laparoscopy) insemination (ITT).

Intra-cervical artificial insemination and intrauterine artificial insemination (IUI). Approximately 85% of artificial insemination procedures performed in the industry use conventional artificial insemination. Intra-cervical artificial insemination requires approximately 2.5×10⁹ sperm cells to 4×10⁹ sperm cells per dose, which is deposited in the posterior part of the cervix. Intra-cervical artificial insemination is not efficient because approximately 90% of the inseminated sperm cells are destroyed before they reach the uterus. This loss of sperm cells is caused by the backflow of semen, and intensive uterine phagocytosis. Intrauterine insemination (IUI) requires about 1.5×10⁹ sperm cells to 1.5×10⁹ sperm cells per dose. Here, the sperm cells are deposited after the cervical canal and into the uterine body to avoid the semen backflow issue. However, these concentrations of sperm cells needed are much higher than the concentrations of sex-sorted sperm cell samples obtained using the techniques described above. During sorting, sperm cell samples are highly diluted, processed, centrifuged, and reconstituted to increase their density. This process affects sperm cell sample volume, the number of viable sperm cells, and the fertilizing ability of sex-sorted sperm cells. It is therefore necessary to use an insemination technique that deposits sex-sorted sperm cells closer to the ovulation and fertilization sites, such as the uterine horn, or the utero-tubal junction. For these reasons, deep intrauterine insemination (DIUI), and intratubal (laparoscopy) insemination (ITI) are preferred techniques for sex-sorted sperm cell insemination.

Deep intrauterine artificial insemination (DIUI). DIUI is performed by depositing sperm cell samples containing approximately 50-900×10⁶ sperm cells per dose in the upper (anterior) third of the uterine horn or at the at the utero-tubal junction. Because of its length, a deep intrauterine catheter allows the operator to reach distal regions of a sow's reproductive tract, including the uterine horns—regions that would be unreachable using a standard artificial insemination catheter. The use of reduced sperm cell doses is possible because the uterine horn and the utero-tubal junction are key areas of the sow's reproductive tract. Methods for carrying out DIUI are known to those skilled in the art. See e.g., U.S. Patent Application Publication No. US 2002/0072650 A1; Martinez et al., Reproduction 123:163-170, 2002; Martinez et al., Reprod. Supp. 58:301-311, 2001; and International Patent Application Publication No. WO 1999/027868, each of which is herein incorporated by reference.

In some embodiments, the methods of the present technology comprise introducing a deep intrauterine catheter comprising the sex-sorted sperm cell sample inside the cervical duct of a sow in estrus. In some embodiments, the sex-sorted sperm cells are deposited in the anterior half of one or both uterine horn(s) near the utero-tubal junction. In some embodiments, the variable estrus cycle of the target sow is hormonally synchronized to ensure that the timing of the DIUI is optimized to maximize the efficiency of insemination. In some embodiments, the variable estrus cycle of the target sow is synchronized by injecting the target sows intramuscularly with 1250 IU equine chorionic gonadotrophin (eCG; Folligon, Intervet International B.V., Boxmeer, The Netherlands). Target sows are determined to be in estrus when they exhibit prolonged muscle contractions known as a “standing reflex.” In some embodiments, a sex-sorted sperm cell sample for DIUI contains about 10×10⁶ sperm cells or less. In some embodiments, a sex-sorted sperm cell sample for DIUI contains at least about 5×10⁶, at least about 10×10⁶, at least about 20×10⁶, at least about 50×10⁶, at least about 100×10⁶, at least about 125×10⁶, at least about 200×10⁶, or at least about 500×10⁶ sperm cells.

Intratubal (laparoscopic) artificial insemination (ITI). Intratubal laparoscopic artificial insemination is a surgical procedure performed by depositing sperm cell aliquots containing approximately 1×10⁶ sperm cells in the oviduct, the isthmus, ampulla, or the utero-tubal junction in the target sow's reproductive tract. Laparoscopic inseminations are performed on sows sedated by azaperone administration (Stresnil; 2 mg/kg body weight, i.m.). General anesthesia is induced with sodium thiopental (Abbot; 7 mg/kg body weight, i.v.) and maintained with halothane (3.5-5%). In some embodiments, a sex-sorted sperm cell sample for intratubal laparoscopy contains about 5×10⁶ sperm cells or less. In some embodiments, a sex-sorted sperm cell sample for intratubal laparoscopy contains at least about 0.5×10⁶, at least about 1×10⁶, at least about 1.5×10⁶, at least about 3×10⁶, at least about 100×10⁶, or at least about 125×10⁶ sperm cells.

In some embodiments, a sex-sorted sperm cell sample of a known, assessed quality may be used to inseminate a target sow herd by artificial insemination. In some embodiments, the sex-sorted sperm cells may be used shortly after the preparation of the sex-sorted sperm cell sample, such as, for example, within about 120 hours, within about 96 hours, within about 72 hours, within about 48 hours, or within about 24 hours after formation of the sperm dispersion. Prior to use in artificial insemination, to ensure the insemination will yield the desired results, the quality and/or efficacy of the sex-sorted sperm cells may be assessed, as described herein. In some embodiments, a sex-sorted sperm cell sample that will be used for insemination comprises at least about 60%, at least about 65%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, or at least about 97% sex-sorted sperm cells. In some embodiments, a sex-sorted sperm cell sample comprises at least about 60%, at least about 65%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, or at least about 97% sperm cells that bear an X-chromosome (and do not bear a Y-chromosome). In some embodiments, a sex-sorted sperm cell sample comprises at least about 60%, at least about 65%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, or at least about 97% sperm cells that bear a Y-chromosome (and do not bear an X-chromosome).

In some embodiments, sex-sorted sperm cells may not have been cryopreserved prior to insemination. Instead, the sex-sorted sperm cells may have been maintained in a motility inhibitor and/or may have been refrigerated at temperatures ranging from about 4° C. to about 25° C., about 10° C. to about 25° C., or about 15° C. to about 20° C. In some embodiments, the sex-sorted sperm cells have been refrigerated at about 18° C. In some embodiments, the sex-sorted sperm cells may be cryopreserved and thawed prior to insemination (i.e., the dispersion is frozen/thawed or comprises frozen/thawed sperm cells). Typically, in such an instance, the cryopreserved sex-sorted sperm cells are thawed immediately, such as, for example, within about 15 minutes, before insemination. In some embodiments, the cryopreserved sex-sorted sperm cells may be thawed over a period of time or thawed and subsequently stored for a period of time, such as, for example, less than about 5 days, less than about 2 days, less than about 1 day, or less than about 12 hours.

C. Use at the Multiplication Unit to Skew Maternal and Paternal Lines

In some embodiments, the present technology provides methods for using sex-sorted sperm cell samples to improve female swine breeding programs for use in commercial farms.

Swine breeding programs use a multi-generational pyramid breeding program to produce crossbred females that are a combination of two or more purebred lines. The swine production pyramid is subdivided into categories of swine herds as follows. The base of the pyramid comprises a commercial herd, followed by a multiplier herd, a daughter nucleus herd, and is capped at the top by a genetic nucleus herd. In some embodiments of the present technology, genetic nucleus female purebred lines are mated to boars of another maternal genetic line to produce crossbred females (daughter nucleus). The crossbred females may in turn be mated to boars of a third maternal genetic line to produce another female swine that may be used to produce commercial swine product for market.

The genetic nucleus herd comprises purebred lines that are used as a breeding supplier stock comprising a number of desirable traits. In some embodiments, sex-sorted sperm cell samples from a single elite boar from the genetic nucleus are used to inseminate all females in the target herd (e.g., daughter nucleus and/or multiplier herd) for an entire generation. In some embodiments, sex-sorted sperm cell samples from either the same or a different genetic nucleus boar are used from one generation to the next. In some embodiments, sex-sorted sperm cells from a genetic nucleus boar are used to inseminate a target sow herd. In some embodiments, sex-sorted sperm cells in which at least about 60%, at least about 65%, at least about 70%, at least about 75%, at least about 80%, at least about 85%, at least about 90%, at least about 95%, at least about 96%, at least about 97%, at least about 98%, or at least about 99% of the sperm cells bear X-chromosomes are used to inseminate a target sow herd to increase the likelihood of producing a higher percentage of female offspring.

Using sex-soiled porcine sperm cells to increase the female birth ratio can yield a number of benefits to producers. There are a number of economic advantages associated with raising female pigs, including improving efficiencies in the finishing barn and eliminating split-sex feeding, for example. Besides the economic benefits, the use of sex-sorted porcine sperm cells to increase the female birth ratio can provide animal welfare benefits at all levels of pig production associated with reducing or eliminating castration of male pigs.

D. Enhanced Performance Trait Dissemination

1. Desirable Phenotypic Traits

The present technology provides for the use of sex-sorted porcine sperm cells to efficiently disseminate a given desirable genetic trait from one generation to the next, such that future generations express the trait at a higher frequency than the parent or grandparent generation. In some embodiments, the present technology provides for the use of sex-sorted sperm cell samples for the simultaneous improvement of skewed maternal lines at the multiplication unit of a breeding program and the dissemination of improved growth performance and pathogen resistant traits for commercial line breeders.

In some embodiments, the breeding boars are selected based on the presence of desirable traits or markers. In some embodiments, the desirable traits or markers comprising one or more of the presence or absence of specific genes and/or alleles, health traits, reproduction traits, meat quality traits, efficient growth traits, qualitative traits, or quantitative traits. Once identified, sex-sorted sperm cell samples from boars possessing the desirable traits and/or markers are used according to the methods described herein. In some embodiments, the desired trait is a single gene, allele, or locus. In some embodiments, the desired trait is fixed in the population within a few generations.

In some embodiments, the methods of the present technology comprise disseminating a desirable trait in a target herd to homozygosity in four generations or less. In some embodiments, the desirable trait is disseminated in the target herd population in two or three generations.

In some embodiments, the methods of the present technology comprise: (a) selecting a desired trait for which improvement is desired; (b) artificially inseminating target sows using sex-sorted sperm cells from an elite boar, wherein said elite boar comprises the desired trait; (c) producing offspring; (d) identifying those offspring that are either heterozygous or homozygous for the desired trait; and (e) retaining as breeding stock those animals which are heterozygous or homozygous for the desired trait.

In some embodiments, the inseminated target sow is a purebred target sow that is of the same genetic line as the boar from which the sex-sorted sperm cell sample was obtained for transferring desired genes/traits to the next generation of sows. In some embodiments, the sow offspring are purebred sow offspring that in turn become ancestors of parent sow for the commercial lines through one or more successive generations of crossing to boars of other maternal genetic lines. In some embodiments, the sow offspring become ancestors of a parent sow for the commercial lines through one or more successive generations by inseminating the purebred sow offspring with the sex-sorted sperm cell sample from a boar of different maternal genetic lines.

In some embodiments, the inseminated target sow is not of the same genetic line as the boar from which the sex-sorted sperm cell sample was obtained for transferring genes/traits to the next generation of sow. In some embodiments, the offspring are crossbred sow offspring that are either parent sow for the commercial line or will become ancestors of parent sow through one or more successive generations of crossing to boars of other maternal genetic lines. In some embodiments, the sow offspring become ancestors of a parent sow for the commercial lines through one or more successive generations by inseminating the purebred sow offspring with the sex-sorted sperm cell sample from a boar of different maternal genetic lines.

In some embodiments, the desired trait is associated with the absence of undesirable physical abnormalities or defects. In some embodiments, the undesirable physical abnormalities or defects are associated with porcine stress syndrome, Rendement Napole (RN), and any other diseases. In some embodiments, the desired trait provides resistance to specific diseases, specific pathogenic organisms, and/or general resistances to pathogens. In some embodiments, the desired trait arises from one or more mutations in a gene disclosed in Table 1. In some embodiments, the one or more mutations are substitutions, insertions, or deletions that revert the disease causing variants to wild-type, and/or non-defective variants. In some embodiments, the one or more mutations are substitutions, insertions, or deletions that enhance reproductive quality of the swine, such as improved litter size. In some embodiments, the one or more mutations are substitutions, insertions, or deletions that render swine resistant to specific diseases, specific pathogenic organisms, and/or general resistances to pathogens. In some embodiments, the one or more mutations are substitutions, insertions, or deletions that confer improved growth performance traits on the population. In some embodiments, mutations associated with the undesirable physical abnormalities or defects are eliminated from the population.

TABLE 1
Mutations Affecting Phenotypic Traits

| | | |
|---|---|---|
| Phenotypic Effect | Gene/Marker | Measurement |

Growth Performance

| | | |
|---|---|---|
| Improved Growth | Melanocortin-4 receptor (MC4R) | Increased backfat and |
| Performance | | growth-rate |
| | High Mobility Group AT-Hook | Increased backfat and |
| | 1(HMGA1) | lean growth |
| | cholecystokinin A receptor (CCKAR) | Increase feed intake, |
| | | hunger control and |
| | | obesity |
| | Protein kinase AMP-activated γ 3 | Enhance Meat quality |
| | subunit (PRKAG3) | |
| | insulin-like growth factor 2 (IGF2) | Increased muscle mass |
| | | and decreased backfat |

Pathogen and Disease Resistance

| | | |
|---|---|---|
| Enterotoxigenic E. coli | Mucin-4, | Resistance |
| F4ab/ac | Mucin-13, | |
| | Mucin-20, | |
| | Transferrin receptor (TFRC), | |
| | Tyrosine kinase non-receptor 2 (ACK1) | |
| | UDP-GlcNAc: betaGal beta-1,3-N- | |
| | acetylglucosaminyltransferase 5 | |
| | (B3GNT5) | |
| Enterotoxigenic E. coli | FUT1 | Resistance |
| F18 | | |
| Porcine Stress Syndrome | Ryanodine receptor | Resistance to stress |
| Halothane (HAL) | | |
| Rendement Napole (RN) | Protein kinase AMP-activated γ 3 | pH; non-acidic pork |
| | subunit (PRKAG3) | |
| Porcine Reproductive | Mx1, | Resistance |
| and Respiratory | BAT2, | |
| Syndrome (PRRS) | EHMT2 | |
| resistance | CD163 | |
| | sialic acid binding Ig-like lectin 1 | |
| | (SIGLEC1) | |
| Coronavirus resistance | Aminopeptidase N (ANPEP) | Resistance |
| Influenza infectivity | Acidic nuclear phosphoprotein 32 | Infectivity |
| | family member A (ANP32A), | |
| | ANP32B, transmembrane serine | |
| | protease 2 (TMPRSS2), transmembrane | |
| | serine protease 4 (TMPRSS4) | |

Reproductive Quality

| | | |
|---|---|---|
| Reproductive Traits | Estrogen Receptor (ESR) | Increase Litter size |
| | Erythropoietin Receptor (EPOR) | Increase uterine |
| | | capacity, and |
| | | litter size |

2. Pathogen Resistance Traits

In some embodiments, the desirable genetic trait comprises one or more mutations that confer resistance to a pathogen, an enhanced growth efficiency trait, an enhanced meat quality trait, an enhanced reproductive trait, and/or an improved health trait. In some embodiments, the pathogen is selected from the group consisting of porcine reproductive and respiratory syndrome (PRRS) virus, Escherichia coli F18, Escherichia coli F4, circovirus, swine influenza virus, coronavirus, Mycoplasma hyopneumoniae, and Actinobacillus pleuropneumoniae.

In some embodiments, the present technology provides for the use of sex-sorted sperm cells from a boar carrying one or more mutations in a gene selected from the group consisting of alpha (1,2) fucosyltransferase 1 (FUT1), Mucin 4, myxovirus resistance protein 1 (Mx1), HLA-B associated transcript 2 (BAT2), Euchromatic histone-lysine N-methyltransferase 2 (EHMT2), Aminopeptidase N (ANPEP), Acidic nuclear phosphoprotein 32 family member A (ANP32A), ANP32B, transmembrane serine protease 2 (TMPRSS2), transmembrane serine protease 4 (TMPRSS4), CD163, and sialic acid binding Ig-like lectin 1 (SIGLEC1). In some embodiments, the sex-sorted sperm cells are from a boar that is resistant to infection by porcine reproductive and respiratory syndrome (PRRS) virus, enterotoxigenic Escherichia coli F18, Escherichia coli F4ab, Escherichia coli F4ac, circovirus, swine influenza virus, coronavirus, Mycoplasma hyopneumoniae, and Actinobacillus pleuropneumoniae.

Porcine Reproductive and Respiratory Syndrome (PRRS). Porcine Reproductive and Respiratory Syndrome (PRRS), which is caused by the PRRS virus (PRRSV), is one of the most economically harmful swine diseases. It is a key component of the economically significant Porcine Respiratory Disease Complex (PRDC). PRRS first emerged in the United States and Europe in 1987 and 1990, respectively, and has subsequently spread worldwide. The etiologic agent of PRRS is an enveloped, positive-stranded RNA virus that is a member of the Arteriviridae family, order Nidovirales.

The infection process of the PRRS virus begins with initial binding of the virus to heparan sulfate on the surface of alveolar macrophages, which is followed by PRRS binding to sialic acid binding Ig-like lectin 1 (sialoadhesin; SIGLEC1, CD169 or SN). The virus is internalized via clathrin-mediated endocytosis. In the endosome, the PRRS virus glycoprotein GP2A and GP4 physically interact with porcine CD163, which facilitates the uncoating of the virus. The uncoating releases the viral genome, and initiates the infection cycle. Mutations in Myxovirus resistance protein 1 (Mx1), HLA-B associated transcript 2 (BAT2), and Euchromatic histone-lysine N-methyltransferase 2 (EHMT2) have also been shown to protect swine from the PRRS virus infection.

Current vaccines do not provide satisfactory protection due to strain variation, inadequate stimulation of the immune system, and emergence of virulent PRRS virus strains. There is therefore a need for a PRRS resistant swine line in commercial farms. In some embodiments, the sex-sorted sperm cells for use in the methods of the present technology are from a PRRS virus-resistant boar that carries one or more mutations in CD163, sialic acid binding Ig-like lectin 1 (SIGLEC1), or a combination thereof. In some embodiments, the one or more mutations are substitutions, insertions, or deletions that abolish the gene function. In some embodiments, the present technology provides for the efficient dissemination of the PRRS resistant trait from a genetic nucleus line to commercial farms using sex-sorted sperm cells from a boar inking a functional CD163, sialic acid binding Ig-like lectin 1 (SIGLEC1) protein, or a combination thereof.

Enterotoxigenic Escherichia coli strain. Enterotoxigenic strains of E. coli are responsible for E. coli-induced diarrheas in young pigs. They have two types of virulence factors: 1) fimbriae adhesins, which allow E. coli binding to and the colonization of porcine intestinal epithelial cells, and 2) enterotoxins that cause fluid secretion. F18 and F4 are the two major known fimbriae adhesins. The F18 adhesin has two variants (F18ab and F18ac), while the F4 has three variants (F4ab, F4ac, and F4ad). The F4ac variant is the most prevalent in piglets. The genetic markers for pig resistance or susceptibility to E. coli F4 are Mucin-4, Mucin 13, Mucin 20, the transferrin receptor (TFRC), tyrosine kinase non-receptor 2 (ACK1), and UDP-GlcNAc:betaGal beta-1,3-N-acetylglucosaminyltransferase 5 (B3GNT5). In particular, swine lines carrying MUC4^{GG} and MUC4^{CG} single nucleotide polymorphisms are susceptible to F4 infection, while MUC4^{CC} lines are resistant. In some embodiments, the methods of the present technology comprise the efficient dissemination of enterotoxigenic E. coli resistant swine lines from a genetic nucleus to commercial farms using sex-sorted sperm cell samples from a boar comprising one or more mutations in a gene selected from Mucin-4, Mucin 13, Mucin 20, the transferrin receptor (TFRC), tyrosine kinase non-receptor 2 (ACK1), B3GNT5, or any combination thereof. In some embodiments, the sex-sorted sperm cells are from an enterotoxigenic E. coli-resistant boar that carries one or more mutations in the Mucin-4 gene.

The genetic markers for pig resistance to E. coli F18 are the alpha (1,2) fucosyltransferase 1 (FUT1) and bactericidal/permeability-increasing protein (BPI) genes. Swine breeds carrying FUT1^{AA} single nucleotide polymorphisms are resistant to E. coli F18 infection, while those that carry FUT1^{AG} and FUT1^{AG} single nucleotide polymorphisms are susceptible to infection. In some embodiments, the methods of the present technology comprise the dissemination of E. coli F18 resistant lines by inseminating one or more target sows with a sex-sorted sperm cell sample from a boar carrying the FUT1^{AA} single nucleotide polymorphism. In some embodiments, the boar carrying the FUT1^{AA} single nucleotide polymorphism is resistant to infection by E. coli F18 strain.

Coronavirus. Coronaviruses, highly contagious and widespread viruses known for their distinctive microscopic halos, are responsible for a variety of deadly intestinal diseases in livestock. The alphacoronavirus, transmissible gastroenteritis virus (TGEV), is a source of high morbidity and mortality in neonatal pigs, a consequence of dehydration caused by the infection and necrosis of enterocytes. Aminopeptidase N (ANPEP) is a putative receptor for TGEV in pigs. Studies have shown that ANPEP null pigs do not support infection with TGEV.

Swine Influenza. Swine influenza is a highly contagious viral infection of pigs. Morbidity rates can reach 100% with swine influenza infections, while mortality rates are generally low. The primary economic impact is related to retarded weight gain resulting in an increase in the number of days to reach market weight. Swine influenza is caused by influenza A viruses in the family Orthomyxoviridae. Influenza A viruses are further characterized by subtype by the two major surface glycoproteins, haemagglutinin and neuraminidase (e.g., H1N1). Acidic nuclear phosphoprotein 32 family member A (ANP32A) and ANP32B proteins have been identified as playing fundamental roles in influenza virus replication and host range determination. Transmembrane serine protease 2 (TMPRSS2) and TMPRSS4 have been shown to facilitate the spread of swine influenza.

3. Improved Growth Performance Traits

In some embodiments, the desirable trait for dissemination is an improved growth performance trait selected from the group consisting of enhanced growth efficiency, enhanced meat quality, enhanced reproductive quality, and improved health. In some embodiments, the enhanced growth efficiency trait is selected from the group consisting of increased average daily gain, increased average daily feed intake, increased feed efficiency, reduced back fat thickness, increased muscle mass, increased loin muscle area, and increased carcass lean percentage.

Rendement Napole. Rendement Napole (RN) phenotype economically affects pork meat quality. The presence of the dominant RN⁻ allele in an animal is associated with inferior meat quality attributes. White skeletal muscles from animals carrying the dominant mutation have approximately 70% increase in the glycogen content, and meat characteristics, such as pH, water content, and lean meat content are also affected. Meat from RN animals is acidic. A DNA test is used to classify animals as carriers (RN⁻/RN⁻ or RN⁻/rn⁺) or negative (rn⁺/rn⁺) for the RN phenotype. The causative mutation for the RN⁻ phenotype is a mutation in the protein kinase AMP-activated γ 3 subunit gene (PRKAG3), which encodes the γ 3 isoform of AMP-activated protein kinase (AMPK). The RN⁻ phenotype is caused by a substitution of Arginine to glutamine at position 200 (Arg200Gln) in PRKAG3 that activates the enzyme and increases glucose uptake.

In some embodiments, the methods of the present technology comprise the dissemination of the negative rn⁺/rn⁺ phenotype to homozygosity in one to three generations. In some embodiments, the sex-sorted sperm cell sample is from a boar that comprises one or more mutations in the PRKAG3 gene. In some embodiments, the one or more mutations are substitutions, insertions, or deletions that abolish the dominant RN⁻ phenotype. In some embodiments, the sex-sorted sperm cell sample is from a boar that carries an arginine at position 200 of PRKAG3. In some embodiments, the sex-sorted sperm cell sample is from a boar that carries one or more intragenic mutations within the PRKAG3 gene that abolish the dominant RN⁻ phenotype. In some embodiments, the sex-sorted sperm cell sample is from a boar that carries one or more extragenic mutations that abolish the dominant RN⁻ phenotype.

Porcine Stress Syndrome (PSS). PSS is a hypermetabolic and hypercontractile syndrome triggered by anesthesia or various stressors that cause a sustained increase in sarcoplasmic calcium ion (Ca′). Porcine stress syndrome is characterized by the sudden death of pigs when they are physically stressed. PSS has a beneficial effect on lean percentage, but this is accompanied by poor meat quality attributes and a condition known as pale, soft, exudative (PSE) pork. In addition, PSS affects litter size in females due to the propensity of female PSS carriers to have an increased number of stillborn piglets. The PSS trait is inherited as an autosomal recessive disorder. The causative gene was initially named “halothane” gene because swine with the homozygous recessive genotype (nn) exhibited malignant hyperthermia upon exposure to the gas halothane (2-bromo-2-chloro-1,1,1-trifluoroethane, an inhaled anesthesia). The causative mutation for the PSS is a guanine to thymine substitution at position 1843 of the ryanodine receptor gene, which results in arginine to cysteine substitution at position 615 of the ryanodine receptor (Arg615Cys). In some embodiments, the sex-sorted sperm cell sample is from a boar that is not a PSS gene carrier based on the presence of guanine at position 1843 of the ryanodine receptor gene. In some embodiments, the methods of the present technology comprise the dissemination of the PSS resistant phenotype to homozygosity in one to three generations.

Improved Feeding Behavior and Body Weight. Feeding behavior and body weight are quantitative traits that reflect an animal's potential for growth that can be measured on each animal in the population. The genetic control of feeding behavior and body weight is mediated by the melanocortin-4 receptor (MC4R) gene. In particular, swine lines carrying the major guanine to alanine substitution at position 1426 (G 1426 A; MC4R^{AA}) in the MC4R gene showed increased backfat thickness, carcass weight, moisture, and saturated fatty acid, decreased unsaturated fatty acid, and fluster growth due to greater feed intake. The substitution G1426A polymorphism results in the substitution of aspartic acid at position 298 to asparagine (Asp298Asn). In addition, swine lines carrying the Asp298Asn substitution show lower meat redness and a higher content of saturated fatty acids. However, the wild-type MCR4 gene (MCAR^{GG}) is strongly associated with lower back fat thickness, higher lean meat percentage, slower growth rate and lower feed intake. The allele frequencies of MCR4 differ greatly among pig breeds and lines depending on the economic usage of the lines. For example, the wild type SNP (Asp298) is present at higher frequency in breeds raised for fresh meat production when compared to breeds selected for cured ham and loin production. In some embodiments, the methods of the present technology comprise the dissemination of both MCR4 alleles in different swine lines and the allocation of the line having different genotypes to respective commercial linse. In some embodiments, each allele is disseminated in the genetic nucleus population using the method of the present technology. In some embodiments, each allele is disseminated in atarget sow population using the method of the present technology.

Muscle Mass and Back at Thickness. The phenotypic value of each trait is the result of a combination of both genetic and environmental effects. In swine, fat deposit, muscle mass, lean meat, lean back fat, sow prolificacy, and/or sow longevity, backfat thickness are controlled by a paternally imprinted quantitative trait loci. A polymorphism that is tightly associated with this trait is located within intron 3 of the insulin-like growth factor 2 (IGF2) gene (g.3072G>A). Because the QTL is paternally imprinted, the favorable effect of the QTL allele is only expressed when the QTL allele is inherited from a boar. In particular, offspring that inherit the QTL from the male parent have lean meat, lean back fat, sow prolificacy, and/or sow longevity when compared to a control. In some embodiments, the sex-sorted sperm cell sample is from a boar that carries an insulin-like growth factor 2 single nucleotide polymorphism.

In some embodiments, the methods of the present technology comprise the dissemination of additional genes that are associated with backfat trait such as the fatty acid synthase (FASN), calpastatin (CAST), high mobility group AT-hook 2 (HMG2A), and/or cholecystokinin type A receptor (CCKAR) genes.

Fatty acid synthase has significant effects on the fatty acid composition of backfat. Fatty acid synthase also affects the gadoleic acid (C20:1, a mono unsaturated fatty acid) content in backfat. Swine carrying the major polymorphism of the fatty acid synthase gene (FASN^{AA}) have increased backfat thickness, texture values, stearic acid, oleic acid, and poly unsaturated fatty acid content. Two single nucleotide polymorphisms in the calpastatin (CAST) gene (Arg249Lys and Ser638Arg) are associated with meat tenderness. Swine carrying the minor polymorphism, CAST^{AA}, have increased backfat thickness, lowered shear force, palmitoleic acid, oleic acid, and increased stearic acid content. The high mobility group AT-hook 1 (HMG1A) polymorphism is highly associated with backfat and lean growth, and the CCKAR gene is associated with feed intake, hunger control, and obesity.

4. Additional Traits

In some embodiments, the methods of the present technology comprise the dissemination of additional traits from one generation to the next, the presence or absence of which can be determined in live animals using tools known in the art. In some embodiments, the methods of the present technology comprise the dissemination of desirable porcine traits such as those provided in Table 2.

Accordingly, in some embodiments of the present technology, sex-sorted sperm cell samples may be selected based on the presence of desirable characteristics in the boar, including but not limited to: the presence or absence of specific genes and/or alleles, health traits, reproduction traits, meat quality traits, and efficient growth traits. Boars may be selected by any suitable means; for example using computer programs or other means for recording parentage/pedigree and selecting the most suitable pairings. In some embodiments, the computer program uses Optimum genetic contribution theory (OGC), which uses relationships among individuals as weighting factors. See e.g., Oh, Evaluation of Optimum Genetic Contribution Theory to Control Inbreeding While Maximizing Genetic Response, Asian-Australas J. Anim. Sci. 25(3): 299-303 (2012). OGC controls for some of the shortcomings of selection based only on EBV from BLUP evaluations. OGC software functions by: (1) identifying the individual having the best EBV; (2) calculating average relationships (rⱼ⁻) between selected individual(s) and candidate individuals; (3) selecting the individual having the best OGC score with EBV*=EBVⱼ (1-krⱼ) using the weighting factor k; (4) repeating the steps until the number of individuals required are reached; and (5) repeating the process for the opposite sex. The OGC algorithm controls inbreeding by over 47% compared to selection with EBV and maintained consistent increases in selection response for at least 20 generations.

According to the methods of the present technology, selected genetic improvements can be made at any or all levels of swine production. That is, improvements may be made in the commercial swine herd, the genetic nucleus herd, and/or a target herd, either independently or concurrently. These herds can be located and operated on farms internal or external to breeding company facilities (e.g. genetic nucleus, target, and/or swine production herds can be owned and operated at customer locations with genetic expertise supplied by the breeding company).

In some embodiments, the present technology also provides for genetic nucleus herds, target herds, and/or swine production herds that have been produced or genetically modified through the use of the methods described herein.

TABLE 2
Porcine Traits for Selection

| | Trait | | |
|---|---|---|---|
| ID | Acronym | Trait Definition | Measurement |
| 1 | fip | Average daily Food Intake in Purebreds | FIRE feeder - Individual Feed |
| | | (in kilograms) | Intake |
| 2 | bfp | BackFat thickness in Purebreds (in mm) | Real-time ultrasound |
| 3 | ldp | Loin Depth in Purebreds (in mm) | Real-time ultrasound |
| 4 | msp | Muscle Score in Purebreds (subject | Visual assessment |
| | | scoring 1 . . . 9) | |
| 5 | lgp | Leg score in Purebreds (subject Scoring | Visual assessment |
| | | system front and back legs) | |
| 6 | afp | Age of the sow at First farrowing in | Herd recording system |
| | | Purebreds | (calendar) |
| 7 | btp | Number of pigs Born Total in Purebreds | Visual assessment |
| | | (count) | |
| 8 | ttp | Number of Teats in Purebreds (count) | Visual assessment |
| 9 | wlp | Weight of the Litter at weaning in | Scale |
| | | Purebreds | |
| 10 | spp | Survival Percent at birth in Purebreds | calculation from visual |
| | | (birth to wean hht) | assessment |
| 11 | shp | Scrotal Hernia in Purebreds | Visual assessment |
| 12 | bsp | Percentage of pigs Born Stillborn in | calculation from visual |
| | | Purebreds | assessment |
| 13 | wrp | Wean to First Mating Interval Groups | Herd recording system |
| | | 1 = 0 to 7 | (calendar) |
| | | 2 = 8 to 40 days | |
| 14 | wsp | Wean to First Mating Interval in days 0 to | Herd recording system |
| | | 40 | (calendar) |
| 15 | bfx | BackFat thickness in Xbreds | Packing plant via Fat-o-Meter |
| 16 | ldx | Loin Depth in Xbreds | Packing plant via Fat-o-Meter |
| 17 | rgp | Ridgling in Purebreds | Visual assessment |
| 18 | rgx | Ridgling in Xbreds | Visual assessment |
| 19 | shx | Scrotal Hernia in Xbreds | Visual assessment |
| 20 | sgp | Splay Legs Purebreds | Visual assessment |
| 21 | sgx | Splay Legs Xbred | Visual assessment |
| 22 | uhp | Umbilical Hernia Purebreds | Visual assessment |
| 23 | upx | Umbilical Hernia Xbreds | Visual assessment |
| 24 | wux | Weight of the boston butt in Xbreds (TS) | Physical weighing at plant |
| 25 | dpx | Death of Pre Nursery in Xbreds | Visual assessment |
| 26 | dnx | Death of Nursery pig in Xbreds | Visual assessment |
| 27 | dfp | Death of the Finisher pig in Purebreds | Visual assessment |
| 28 | dfx | Death of the Finisher pig in Xbreds | Visual assessment |
| 29 | dpp | Death of the Pre Nursery pig in Purebreds | Visual assessment |
| 30 | dnp | Death of the Nursery pig in Purebreds | Visual assessment |
| 31 | whx | Ham bone-in wt Xbreds | Physical weighing at plant |
| 32 | wmx | Weight of the boneless ham in Xbreds | Physical weighing at plant |
| 33 | wox | Weight of the bone-in Loin in Xbreds | Physical weighing at plant |
| 34 | wnx | Weight of the boneless loin in Xbreds | Physical weighing at plant |
| 35 | wbx | Weight of the Belly in Xbreds | Physical weighing at plant |
| 36 | phx | Loin pH in Xbreds | pH probe at slaughter facility |
| 37 | pax | pH Ham Xbreds | pH probe at slaughter facility |
| 38 | wdx | Weight of the cold Carcass in Xbreds | Physical weighing at plant |
| 39 | mix | Loin Minolta L in Xbreds | Minolta at slaughter facility |
| 40 | glp | Average daily Gain over the Lifetime in | Calculation from physical |
| | | Purebreds (in grams) | weight at farm |
| 41 | glx | Average daily Gain over the Lifetime in | Calculation from physical |
| | | Xbreds (in grams) | weight at farm |
| 42 | gcx | Average daily Gain of the hot Carcass | Calculation from physical |
| | | weight over the lifetime in Xbreds (in | weight at slaughter facility |
| | | grams) | |
| 43 | wix | Weight of the picnic shoulder in XBreds | Physical weighing at plant |
| | | (TS) | |
| 44 | wpx | Weight of the spare rib in XBreds (TS) | Physical weighing at plant |
| 45 | uip | Ultrasound Intramuscular fat in Purebreds | Real-time ultrasound |
| 46 | lcp | Lactate Level after 100 m (330 ft) Fitness | Stress test with lactate |
| | | in Purebreds (mmol/l) | measurement |
| 47 | WBS | Warner-Bratzler shear force (WBS) pork | Assessed on cut of meat |
| | | tenderness | |

E. Developing Specialized Pork Products and Niche Pork Markets

In some embodiments, sex-sorted sperm cell samples are used in methods for furthering genetic lines with superior pork qualities as assessed by the following characteristics, which include but are not limited to, pH, visual color, intramuscular fat, water holding capacity (WHC), increased marbling, or flavor of the pork. In some embodiments, sex-sorted sperm cell samples can be used in methods for developing production flows for specialized pork products characterized by any one or more of these desirable qualities. For example, the methods of the present technology can be employed to advance genetic-based niche marketing programs and to meet the needs of producers selling to niche markets where there is a demand for one or more unique and/or desirable characteristics in the pork product (e.g., high marbling pork, pork flavor, etc.).

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Phenotypic Selection of Swine Line Having Desirable Traits

This example will demonstrate the selection of desirable traits for dissemination in a breeding program.

Breeding will be initiated using parentage derived from the Pietrain, Landrace, Large White and Duroc breeds, although breeding may be initiated in any type of desired breed. The breeding objective will be made up of several economically important traits including growth rate, feed intake, and last rib backfat. Growth rate and backfat will be measured at approximately 160 days of age using electronic scales and ultrasound. Feed intake will be measured as individual animal feed consumption through the use of electronic feeding equipment. The measured traits, along with the pedigrees and genotype information will be used to compute Estimated Breeding Values (EBV). These EBV will then be weighted by their associated economic values resulting in a computed index for every swine that is a selection candidate. The index then becomes the objective genetic description of profit potential for each selection candidate.

Semen will be collected from selected boars with the desired EBV and prepared according to methods known in the art. A typical boar ejaculates 70-100 billion cells, which will be subdivided into 2.5 billion sperm cells per dose in 70 ml. These doses will produce enough semen to mate approximately 150 to 200 sows/year. The selected sows will be inseminated using intrauterine artificial insemination twice about 24 hours apart. See Example 3 infra. Good sows will have a farrowing rate of at least 92%. In addition to the selected traits, sex-sorted sperm cells comprising at least 65% to 90% X-chromosome bearing sperm cells will be used for artificial insemination.

It is predicted that the selected trait is recessive and has an intermediate frequency of about 0.5 in the population, which means that about 25% of the population carries the desired trait. It is predicted that artificially inseminating target sows with sex-sorted sperm cells from an elite boar having a desired trait at the same frequency of 0.5 as the target sows will produce a population of mostly female offspring having the selected trait in about 50% of the animals. Therefore, the methods of the present technology will double the frequency of the desired traits in about one generation. The female progeny carrying the homozygote recessive allele will be used as replacement sows for the target herd, and will be artificially inseminated using sex-sorted sperm cells from a boar from a different boar line also carrying homozygote recessive allele at the same frequency. It is predicted that the desired trait would be in 100% of the population in two generations. If however, the frequency of the desired trait is about 0.05, it is expected that in the first generation, 5% of the offspring will have the homozygote recessive allele, and 52.2% will have the desired trait in the second generation.

It is predicted that at least 75% of the progeny from each cross will be female. It is further predicted that the offspring will have a growth rate ranging between at least about 509.4 to about 1028.1 g; a backfat ranging between at least about 5.1 to about 17.8 mm; and a feed intake ranging from about 1.4 to about 2.8 kg in about one to two generation. It is also predicted that the EBV index for the top 10% of the selected boars will be about 146.85, while the EBV index for the bottom 10% will be about 107.0. It is further predicted that artificial insemination using sex-sorted sperm cells will lower swine production cost.

Accordingly, these results will demonstrate that artificial insemination using sex-sorted sperm cells is a quick and efficient method for enhancing the dissemination of desirable phenotypic traits in a porcine herd or population.

### Example 2: Identification of Pathogen Resistant and Improved Growth Performance Trait Genetic Markers in Offspring

This example will demonstrate methods for determining the genotype of a particular breed line following artificial insemination.

Several methods exist to determine genotype of a selected swine breed. Such methods include, but are not limited to: PCR amplification and sequencing using suitable primer pairs consisting of DNA sequence flanking the polymorphism of interest; RFLP analysis using suitable primers flanking the polymorphism in conjunction with a restriction endonucleases that discriminate between the desired alleles in the amplified DNA; real time PCR analyses involving DNA amplification and probe hybridization where the hybridization probes are labeled and discriminate between the allelic forms; and other methods readily performed by those skilled in the art.

In particular, the swine breeds will be genotyped using commercially available genotyping chips and corresponding relationship based genomic algorithm and software, such as Illumina PorcineSNP60 BeadChip. DNA from at least 14 individuals will be extracted using Qiagen DNeasy Tissue kit (Qiagen, Germany). All DNA samples will be analyzed by spectrophotometry and agarose gel electrophoresis. The genotyping platform will be performed with Infinium II Multisample assay (Illumina SNP arrays will be scanned using iScan (Illumina Inc.) and analyzed with BeadStudio (Version 3.2.2, Illumina, Inc.). The SNPs physical positions on chromosomes will be derived from the swine genome sequence assembly (10.2) (ensembl.org/Sus_scrofa/Info/Index). The SNPs that will not be mapped on the Chip, or those that will be mapped to multiple positions in the Sscrofa10.2 assembly will be evaluated using traditional techniques known to the art as disclosed above.

Quantitative real time PCR (qPCR) will be used to validate identified polymorphism. The glucagon gene (GCG), which is highly conserved between swine species will be used as an internal control. All qPCR will be carried out using LightCycler® 480 SYBR Green I Master on Roche LightCycler® 480 instrument following the manufacturer's guidelines and cycling conditions. The reactions will be carried out in a 96-well plate in 20 μl volume, containing 10 μl Blue-SYBR-Green mix, 1 μl forward and reverse primers (10 pM/μl) And 1 μl 20 ng/μl genomic DNA. Animals having the correct genotype will be selected for artificial insemination and breeding.

It is predicted that the allele frequency for pathogen resistance and improved growth rate will be 100% homozygous in about two to three generations using the method of the present technology.

Accordingly, these results will demonstrate that artificial insemination using sex-sorted sperm cells, when combined with advanced molecular genetic tools and trait selection, can reduce genetic lag in swine production, and is an efficient method for enhancing the dissemination of desirable phenotypic traits in a porcine herd or population.

### Example 3: Reduced Dose Deep Intrauterine and Laparoscopic Artificial Insemination

This example will demonstrate the use of deep intrauterine and laparoscopic artificial insemination using reduced sex-sorted sperm cell doses.

Sex-sorted sperm cells preparation. Insemination using sex-sorted sperm cells will occur shortly after the sorted sperm cell population is obtained, such as for example, within about 24 hours, within about 48 hours, within about 72 hours, within about 96 hours, or within about 120 hours. Prior to use in artificial insemination, the quality and/or efficacy of the sex-sorted sperm cells will be assessed.

To assess the quality and/or efficacy of the sample, sex-sorted sperm cell sample will be centrifuged at a low speed at room temperature for about 5 minutes. The supernatant will be discarded and the resulting sperm cells pellets will be resuspended in a fresh semen extender developed for sex-sorted swine sperms to a final concentration of about 10×10⁶ sperm cells per milliliter. The concentration will be determined using the NucleoCounter® SV-100™ system (ChemoMetec A/S, Gydevang 43, DK-3450 Allerød, Denmark). The concentrated sex-sorted sperm cells will then be diluted with the same extender and assessed for motility and viability. For all sperm cells quality evaluations, 25×75 mm glass microscope slides (Andwin) and 22×22 mm #1.5 coverslips (Thomas Scientific) will be used, and motility assessments will be made using brightfield microscopy. The post intact acrosomes and morphology assessments will be made using differential interference contrast (DIC) microscopy at 400× magnification. Fresh sex-sorted sperm cells that will exhibit a minimum motility of ≥55%, and a primary morphologies ≤15%, secondary morphologies ≤15%, and a total morphology count not to exceed 25% will be selected for artificial insemination. For example, a sex-sorted sperm cell sample will be tested to confirm that the sample comprises at least 75%, at least 80%, at least 85%, at least 90%, or at least 95% X-chromosome or Y-chromosome bearing sperm cells. For the production of female offspring, samples in which sperm cells bear at least 75%, at least 80%, at least 85%, at least 90%, or at least 95% X-chromosome will be selected.

Deep intrauterine artificial insemination. Deep intrauterine insemination will be carried out using a sex-sorted sperm cell sample comprising about between about 2×10⁴ to about 4×10⁶ sperms per dose in a volume of about 5 ml to about 20 ml per dose. Sows exhibiting “standing reflex” will be selected for insemination. The catheter will be lubricated using a non-toxic liquid lubricant, and will be inserted through the vagina using a flexible probe until it reaches its final position within the uterine horn. Once at that position, a syringe containing the sex-sorted sperm cell sample will be introduced into the uterine environment through a flexible duct found within the flexible probe of the catheter. Another syringe containing a small volume of buffer will be applied through the flexible duct to ensure the complete evacuation of the sex-sorted sperm sample in the uterine horn.

Laparoscopic artificial insemination. Laparoscopic artificial insemination will be performed on sedated sows using laparoscopic techniques known in the art. A sex-sorted sperm cell sample containing 0.1-0.5×10⁶ sperm cells in 100 microliter will be flushed directly into the oviduct, the isthmus, the ampulla, or the utero-tubal junction.

It is anticipated that deep intrauterine insemination and laparoscopic artificial insemination will result in at least about 75-90% pregnancy rate with at least 95% viable embryos for each inseminated sow.

Accordingly, these results will demonstrate that the sex-sorted porcine sperm samples when combined with deep intrauterine insemination of the present technology are useful in methods for enhancing the dissemination of desirable phenotypic traits in a porcine herd or population

### Example 4: Producing a Porcine Reproductive and Respiratory Syndrome (PRRS) Virus-Resistant Porcine Herd Using Deep Intrauterine Artificial Insemination

This example will demonstrate the generation of PRRS-resistant porcine herd using deep intrauterine artificial insemination.

Boars carrying a non-functional or inactivated CD163 or CD169 gene from a genetic nucleus herd will be identified using the methods of Example 2. These boars are known to those skilled in the art. See e.g., U.S. Pat. Nos. 9,854,790 and 10,405,526. Four heterozygous or homozygote boars will be selected as male founders (F0 generation). The boars will also be selected based on age and EBV value. Semen will be collected from the four boars and sex-sorted sperm cells will be prepared using well-known methods in the art. 30 sows will be selected based on the presence of a functional CD163 or CD169 gene using the methods of Example 2. The frequency of the non-functional allele is 0.5, and currently, about 25% of the population bears the non-functional alleles of CD163 or CD169. The sows will also be grouped based on their EBV and age, and will be inseminated using the deep intrauterine artificial insemination of Example 3. Sows are expected to produce about eight offspring each. The selected boars and the target sows will have the same frequency for the CD163 or CD169 non-functional allele. It is predicted that 50% of the F₁ offspring will carry the PRRS-resistant allele.

Female F₁ offspring will be artificially inseminated using sex-sorted sperm cells from a PRRS-resistant boar from a different boar line also carrying homozygote recessive non-functional alleles of CD163 or CD169 at the same frequency. It is predicted that the PRRS-resistant trait would be in 100% of the F₂ population. If however, the frequency of the PRRS-resistant trait is about 0.05, it is expected that in the F₁ generation, 5% of the offspring will be homozygote recessive for the nonfunctional CD163 and CD169 alleles, and 52.2% will have the PRRS-resistant in the second generation.

Accordingly, these results will demonstrate that artificial insemination using sex-sorted sperm cells when combined with advanced molecular genetic tools and trait selection is an efficient method for enhancing the dissemination of PRRS-resistant traits in a porcine herd or population.

### Example 5: Increasing the Number of Female Offspring at the Multiplication Unit

This example will demonstrate the production of skewed maternal lines at the multiplication unit using deep intrauterine artificial insemination.

Breeding will be initiated using parentage derived from the Pietrain, Landrace, Large White and Duroc breeds. The breeding objective will be made up of several economically important traits including enhanced growth, enhanced meat quality, enhanced reproductive quality, and improved health. 132 target sows will be selected and inseminated with a sex-sorted sperm cell population comprising about 2×10⁴ to about 4×10⁶ X-bearing sperm cells. 50 target sows will be inseminated with the same dose of non-sorted sperm cells. The pregnancy rate will be examined twice during parturition. It is expected that the pregnancy rate will about 38% to 60% with sex-sorted sperm cells and 70%-80% for unsexed sperm cells. It is anticipated that the sex ratio of the progeny will be at least 85.3% female in animals inseminated with sex-sorted sperm cells and 50-58.6% in non-sorted sperm cells.

Accordingly, these results will demonstrate that artificial insemination using sex-sorted sperm cells is an efficient method for increasing the population of females in a porcine herd or population.

### Example 6: Production of Sexed Porcine Semen

This example demonstrates the production of sexed porcine semen for use in, for example, producing a pathogen-resistant porcine herd or population, for increasing the number of female offspring at a multiplication unit, or for other uses.

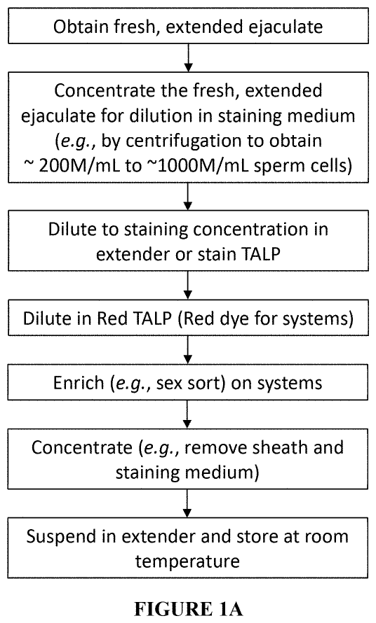

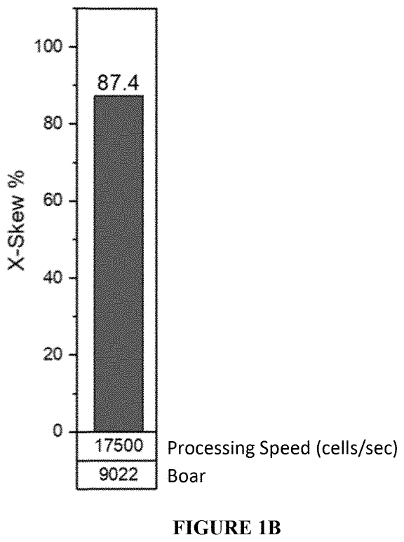

A schematic outlining the general method employed in this Example for the production of a sex-sorted porcine sperm cell sample is provided in FIG. 1A. Briefly, fresh, extended ejaculate samples from a boar were received and evaluated to identify the motility, concentration, and morphology of the samples. Unextended fresh ejaculate typically has a cell concentration of approximately 20 M/mL, and the extended samples received had a cell concentration of approximately 1000 M/mL. Motility at all stages of processing was determined using an IVOS reader, and fresh sample motility was at approximately 90% progressively motile cells. The extender used may be ELIXIR (GenePro, Inc., Fitchburg, Wis.), ANDROSTAR (Minitube USA, Inc., Verona, Wis.), or NUTRIXcell+(IMV Technologies, Maple Grove, Minn.); however, the method described herein is not intended to be limited by the type of semen extender that is used.

The concentrated, extended samples were then diluted and stained using a stain TALP comprising Hoechst 33342. A red dye TALP was then added to the stained samples. Although a stain TALP/red TALP staining media was used in this Example, a semen extender may also be used as a staining medium. The stained sample had a cell concentration of approximately 200 M/mL. After staining, the sample was enriched on a flow cytometer.

The stained samples were processed by flowing the sample through a microfluidic chip at a rate of 17,500 cells/sec. The sample was interrogated by a first source of electromagnetic radiation, and a fluorescence of each cell was detected by a detector. Based on a difference in DNA content between X-chromosome and Y-chromosome bearing sperm cells, a second source of electromagnetic radiation was used to ablate or slice Y-chromosome bearing sperm cells. A sample tube was used to collect the sample which had been enriched for X-chromosome bearing sperm cells. A progressive motility of approximately 60% was observed after enrichment. The enriched or sexed ejaculate samples from the boar (“Boar 9022” in this example) were pooled and spun down in 250 mL conical flasks to pelletize the samples. The samples were then aspirated to approximately 1 mL, and 1 mL of semen extender was then added to the aspirated samples to resuspend the pellet. This process formed extended samples comprising approximately 25 million cells/mL.

A glass-wool procedure was used to separate motile sperm cells from dead or damaged sperm cells, and ddPCR was used to determine the skew of the separated, motile sperm cells. As shown in FIG. 1B, the skew of the enriched sample was determined to be approximately 87%.

Accordingly, these results demonstrate the production of a viable, sexed porcine semen for use in, for example, producing a pathogen-resistant porcine herd or population, for increasing the number of female offspring at a multiplication unit, or for other uses.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for producing a pathogen-resistant porcine herd or population, comprising: inseminating one or more target sows with a sex-sorted sperm cell sample from a boar, wherein the boar comprises one or more pathogen-resistant markers, thereby producing offspring comprising one or more pathogen-resistant markers.

2. The method of claim 1, further comprising: inseminating one or more females from the offspring with a sex-sorted sperm cell sample from a boar having one or more pathogen-resistant markers, wherein the one or more pathogen-resistant markers from the boar and the one or more female offspring are the same or different.

3. The method of claim 2, wherein the one or more female offspring is a member of a daughter nucleus line or a multiplier line, and the boar is a member of a genetic nucleus line.

4. (canceled)

5. The method of claim 2, wherein the boar or the one or more target sows are a member of a genetic nucleus line, a daughter nucleus line or a multiplier line.

6. The method of claim 1, further comprising:
wherein the pathogen-resistant porcine herd or population is a pathogen-resistant female porcine herd or population;
wherein the boar is an elite boar;
wherein at least 60% of the sperm cells in the sex-sorted sperm cell sample carry X chromosomes; and
wherein about 65% to about 99% of the offspring are female, and the female offspring comprise the one or more pathogen-resistant markers.

7. The method of claim 6, further comprising:
inseminating one or more females from the offspring with a sex-sorted sperm cell sample from the elite boar having one or more pathogen-resistant markers, wherein the one or more pathogen-resistant markers from the elite boar and the one or more female progeny are the same or different, and wherein at least 60% of the sperm cells in the sex-sorted sperm cell sample carry X chromosomes.

8. The method of claim 1, wherein, the pathogen-resistant herd or population is resistant to a pathogen selected from the group consisting of porcine reproductive and respiratory syndrome (PRRS) virus, Escherichia coli F18, Escherichia coli F4ab, Escherichia coli F4ac, circovirus, swine influenza virus, coronavirus, Mycoplasma hyopneumoniae, and Actinobacillus pleuropneumoniae.

9. (canceled)

10. The method of claim 1, wherein the one or more pathogen-resistant markers comprise one or more mutations in a gene selected from the group consisting of alpha (1,2) fucosyltransferase 1 (FUT1), Mucin 4, myxovirus resistance protein 1 (Mx1), HLA-B associated transcript 2 (BAT2), Euchromatic histone-lysine N-methyltransferase 2 (EHMT2), Aminopeptidase N (ANPEP), Acidic nuclear phosphoprotein 32 family member A (ANP32A), ANP32B, transmembrane serine protease 2 (TMPRSS2), transmembrane serine protease 4 (TMPRSS4), CD163, and sialic acid binding Ig-like lectin 1 (SIGLEC1).

11. (canceled)

12. The method of claim 1, wherein the sex-sorted sperm cell sample from the boar comprises a combination of sex-sorted sperm cells from one or more boars having one or more pathogen-resistant markers.

13. (canceled)

14. A method of enhancing the dissemination of improved growth performance traits in a porcine herd or population, comprising:
(a) obtaining a sperm cell sample from a boar, wherein the boar comprises one or more improved growth performance traits selected from the group consisting of: enhanced growth efficiency, enhanced meat quality, enhanced reproductive quality, and improved health;
(b) enriching the sperm cell sample obtained from the boar;
(c) inseminating one or more target sows with the enriched sperm cell sample; and
(d) producing offspring having the one or more improved growth performance traits.

15. The method of claim 14, further comprising:
(e) selecting one or more female offspring having the one or more improved growth performance traits that would enhance the dissemination of the one or more improved growth performance traits to the next generation; and
(f) inseminating the selected one or more female offspring with an enriched sperm cell sample that was obtained from a sperm cell sample of a boar having one or more improved growth performance traits, wherein the one or more improved growth performance traits from the boar and the one or more female offspring are the same or different.

16. The method of claim 14, wherein the enhanced growth efficiency trait is selected from the group consisting of increased average daily gain, increased average daily feed intake, increased feed efficiency, reduced back fat thickness, increased muscle mass, increased loin muscle area, and increased carcass lean percentage.

17. The method of claim 14, wherein the enhanced growth efficiency trait comprises a mutation in a gene encoding a protein selected from the group consisting of ryanodine receptor, protein kinase AMP-activated gamma 3 (AMPKy-3, PRKAG3)), paired-like homeodomain transcription factor 2 (Pitx2), Insulin-like growth Factor 2 (IGF2), high mobility group AT-hook 2 (HMG2A), cholecystokinin A receptor (CCKAR), fatty acid synthase (FASN), calpastatin (CAST 249, 638), and melanocortin-4 receptor (MC4R) gene.

18. The method of claim 14, wherein the improved growth performance trait comprises an enhanced reproductive quality and wherein the enhanced reproductive quality comprises a mutation in a gene encoding a protein selected from the group consisting of estrogen receptor (ER) and erythropoietin receptor (EPOR).

19. The method of claim 14, further comprising:
wherein the porcine herd or population comprises an enhanced female porcine herd or population with one or more improved growth performance traits;
wherein the boar is an elite boar;
wherein the enriched sperm cell sample comprises a sex-sorted sperm cell sample from the elite boar;
wherein at least 60% of the sperm cells in the sex-sorted sperm cell sample carry X chromosomes; and
wherein about 65% to about 99% of the offspring are female, and the female offspring carry the one or more improved growth performance traits.

20. The method of claim 14, wherein the boar or the one or more target sows are a member of a genetic nucleus line, a daughter nucleus line, or a multiplier line.

21. (canceled)

22. The method of claim 15, wherein the one or more female offspring are members of a daughter nucleus line, or a multiplier line, and the boar is a member of a genetic nucleus line.

23. (canceled)

24. The method of claim 14, wherein enriching the sperm cell sample obtained from the boar comprises sexing the sperm cell sample for X- or Y-chromosome bearing sperm cells to obtain a sex-sorted sperm cell sample.

25. The method of claim 24, wherein the sex-sorted sperm cell sample from the boar comprises a combination of sex-sorted sperm cells from one or more boars having one or more improved growth performance traits.

26. (canceled)

27. A method of increasing the number of female offspring in a porcine herd or population, comprising: inseminating one or more target sows with a sex-sorted sperm cell sample from a boar to produce offspring, wherein the boar is a member of a genetic nucleus line, and the one or more target sows are members of a daughter nucleus line or a multiplier line, and wherein about 65% to about 99% of the offspring are female.

28. The method of claim 27, wherein the offspring are terminal parent lines.

29. The method of claim 27, wherein the inseminated one or more target sows produce about 0 to 35% male offspring.

30.-32. (canceled)

33. The method of claim 27, wherein inseminating the one or more target sows comprises administering at least 0.5×10⁶ sex-sorted sperm cells to the reproductive tract of the one or more target sows using intratubal insemination.

34. The method of claim 27, wherein inseminating the one or more target sows comprises administering at least 10×10⁶ sex-sorted sperm cells to the reproductive tract of the one or more target sows using deep intrauterine insemination.

35.-52. (canceled)

53. A method for producing a high health porcine herd or population comprising inseminating one or more sows with a sex-sorted sperm cell sample from a boar that is selected for having at least one health trait to produce a progeny having high health.

54. The method of claim 53, wherein the health trait is selected from one or more of: the absence of an undesirable physical abnormality; improved feet and leg soundness; resistance to specific diseases or disease organisms; or general resistance to pathogens.

55. (canceled)

56. The method of claim 54, wherein the pathogen is selected from one or more of porcine reproductive and respiratory syndrome (PRRS) virus, Escherichia coli F18, Escherichia coli F4ab, Escherichia coli F4ac, circovirus, swine influenza virus, Mycoplasma hyopneumoniae, and Actinobacillus pleuropneumoniae.

57. (canceled)

58. The method of claim 54, wherein the resistance to specific disease organisms is associated with one or more mutations in a gene selected from the group consisting of alpha (1,2) fucosyltransferase 1 (FUT1), Mucin 4, myxovirus resistance protein 1 (Mx1), HLA-B associated transcript 2 (BAT2), Euchromatic histone-lysine N-methyltransferase 2 (EHMT2), Aminopeptidase N (ANPEP), Acidic nuclear phosphoprotein 32 family member A (ANP32A), ANP32B, transmembrane serine protease 2 (TMPRSS2), transmembrane serine protease 4 (TMPRSS4), CD163, and sialic acid binding Ig-like lectin 1 (SIGLEC1).

59.-60. (canceled)

61. The method of claim 53, wherein the sex-sorted sperm cell sample from the boar comprises a combination of sex-sorted sperm cells from one or more boars having at least one health trait.

62. (canceled)
